(19) United States
(12) Patent Application Publication
(10) Pub. No.: US 2026/0097154 A1
(43) Pub. Date: Apr. 9, 2026

(54) EPTFE HIGH STRENGTH SUTURES

(71) Applicant: Zeus Company LLC, Orangeburg, SC (US)

(72) Inventors: Alexandra Younan, Columbia, SC (US); Nancy D. Roberts, Cameron, SC (US); Sean Harley, Orangeburg, SC (US); Shawkat Abdelhaq, Summerville, SC (US)

(21) Appl. No.: 19/349,760

(22) Filed: Oct. 3, 2025

Related U.S. Application Data
(60) Provisional application No. 63/703,488, filed on Oct. 4, 2024.

(51) Int. Cl. A61L 31/04; C08F 14/26; D01D 5/04; D01D 10/02; D01F 6/12

(52) U.S. Cl. CPC A61L 31/048; C08F 14/26; D01D 5/04; D01D 10/02; D01F 6/12; D10B 2321/042; D10B 2401/063; D10B 2509/04

(57) ABSTRACT

Expanded polytetrafluoroethylene (ePTFE) monofilaments with superior knot pull tensile strength are described herein, which can be used as sutures and in medical devices such as for tethers and anchors for cardiac repair. The properties of these monofilaments balance the need for an inert, soft, lubricious and porous material with sufficient tensile properties for high strength applications.

### CROSS-REFERENCE TO RELATED APPLICATIONS

The present application claims priority to and the benefit of U.S. Provisional Application No. 63/703,488, filed on Oct. 4, 2024, the disclosure of which is incorporated herein by reference in its entirety.

### FIELD OF THE INVENTION

The present invention relates to products and, in particular, filaments comprising expanded poly(tetrafluoroethylene) (“ePTFE”). The filaments can be used, e.g., as sutures.

### BACKGROUND

Surgical sutures used in wound closure are made of a variety of materials depending on their application. Each of these materials has its own advantages in certain clinical scenarios based on properties such as ability to knot, uniformity, and tendency of biological reaction. Surgeons generally select a suture with the greatest predictable tensile strength consistent with size limitations, good handling properties, and secure knot tying. Sutures can be made of both absorbable materials (such as gut material and polyglycolic acid) and non-absorbable materials (such as polypropylene, expanded polytetrafluoroethylene (ePTFE), silk, and stainless steel).

ePTFE sutures are of significant interest for certain applications. However, current ePTFE sutures and monofilaments on the market can pose a challenge to applications that require superior tensile properties, e.g., as they do not exhibit sufficient knot pull strengths. It would be advantageous to provide further monofilaments and sutures sufficient for these and other applications.

### SUMMARY

An expanded poly(tetrafluoroethylene) (“ePTFE”) suture or microfilament is provided with enhanced tensile properties, specifically relating to knot pull strength relative to standard ePTFE suture offerings on the market. Such ePTFE sutures and microfilaments can be prepared, e.g., by altering processing conditions during the ram extrusion process such that the tensile properties (e.g., knot pull strength) may advantageously be increased in some embodiments without any secondary processing. In some embodiments, however, the suture may be drawn down following its production to decrease its outer diameter (OD) while maintaining its tensile properties in order to achieve a smaller suture size with enhanced tensile properties.

In some embodiments, the OD of the suture is between 0.0075 inches and 0.031 inches. In some embodiments, the knot pull strength maximum load is up to 14.30 lbf, e.g., between 4.37 lbf and 14.30 lbf. In some embodiments, the density of the suture is between 0.75 and 1.79 g/cc. In some embodiments, the knot pull strength is comparable to the straight pull strength. In some embodiments, the knot pull strength is greater than the straight pull strength. In some embodiments, the knot pull strength is less than the straight pull strength. In some embodiments, the drawdown process increases the knot pull strength.

The disclosure includes, without limitation, the following embodiments:
Embodiment 1: A non-absorbable, monofilament suture comprising expanded polytetrafluoroethylene (“ePTFE”), wherein: the non-absorbable, monofilament suture has a non-contact outer diameter (“OD”) of 0.016 inches or less, and an average knot pull strength in lbf, divided by the OD (“AKP/OD”), of 3884 or greater; and/or the non-absorbable, monofilament suture has a non-contact outer diameter (“OD”) of 0.018 inches or less, and an average straight pull strength in lbf, divided by the OD (“ASP/OD”), of 400 or greater.
Embodiment 2: The non-absorbable, monofilament suture of Embodiment 1, wherein the AKP/OD is 350 or greater.
Embodiment 3: The non-absorbable, monofilament suture of Embodiment 1 or 2, wherein the AKP/OD is 400 or greater.
Embodiment 4: The non-absorbable, monofilament suture of any of Embodiments 1-3, wherein the ASP/OD is 400 or greater.
Embodiment 5: The non-absorbable, monofilament suture of any of Embodiments 1-4, wherein the ASP/OD is 500 or greater.
Embodiment 6: The non-absorbable, monofilament suture of any of Embodiments 1-5, wherein the OD is 0.018 inches or less, and wherein the AKP/OD is 350 or greater and the ASP/OD is 0.40 or greater.
Embodiment 7: The non-absorbable, monofilament suture of any of Embodiments 1-6, wherein the average knot pull strength in lbf is 15 lbf or less.
Embodiment 8: The non-absorbable, monofilament suture of any of Embodiments 1-7, wherein the average knot pull strength in lbf is 14.3 lbf or less.
Embodiment 9: A non-absorbable, monofilament suture comprising expanded PTFE (“ePTFE”), selected from the group consisting of: a non-absorbable, monofilament suture comprising ePTFE with a USP diameter of 5-0 and a knot pull strength of at least 3.0 lbf or at least 3.2 lbf; a non-absorbable, monofilament suture comprising ePTFE with a USP diameter of 4-0 and a knot pull strength of at least 4.8 lbf; a non-absorbable, monofilament suture comprising ePTFE with a USP diameter of 3-0 and a knot pull strength of at least 6 lbf or at least 7 lbf; a non-absorbable, monofilament suture comprising ePTFE with a USP diameter of 2-0 and a knot pull strength of at least 7 lbf or at least 8 lbf; a non-absorbable, monofilament suture comprising ePTFE with a USP diameter of 0 and a knot pull strength of at least 9 lbf, at least 10 lbf, or at least 11 lbf; and a non-absorbable, monofilament suture comprising ePTFE with a USP diameter of 1 and a knot pull strength of at least 13 lbf or at least 14 lbf.
Embodiment 10. The non-absorbable, monofilament suture of any of Embodiments 1-9, wherein the knot pull strength is 15 lbf or less.
Embodiment 11: The non-absorbable, monofilament suture of any of Embodiments 1-10, wherein the knot pull strength is 14.3 lbf or less.
Embodiment 12: A non-absorbable, monofilament suture comprising expanded PTFE (“ePTFE”), selected from the group consisting of: a non-absorbable, monofilament suture comprising ePTFE with a USP diameter of 5-0 and a knot pull strength of at least 3.0 lbf or at least 3.2 lbf; and a non-absorbable, monofilament suture comprising ePTFE with a USP diameter of 3-0 and a knot pull strength of at least 6 lbf or at least 7 lbf.
Embodiment 13: The non-absorbable, monofilament suture of Embodiment 12, wherein the knot pull strength is 15 lbf or less.
Embodiment 14: The non-absorbable, monofilament suture of Embodiment 12 or 13, wherein the knot pull strength is 14.3 lbf or less.
Embodiment 15: The non-absorbable, monofilament suture of any of Embodiments 1-14, consisting essentially of ePTFE.
Embodiment 16: The non-absorbable, monofilament suture of any of Embodiments 1-14, comprising one or more components associated with the ePTFE, wherein the one or more components are selected from the group consisting of agents to improve handling, antibacterial properties, and/or visibility.
Embodiment 17: The non-absorbable, monofilament suture of any of Embodiments 1-16, wherein the suture is uncoated.
Embodiment 18: The non-absorbable, monofilament suture of any of Embodiments 1-17, wherein the non-absorbable monofilament suture has a density of 1.8 g/cc or less.
Embodiment 19: The non-absorbable, monofilament suture of any of Embodiments 1-18, wherein the density is 1.60 g/cc or less.
Embodiment 20: The non-absorbable, monofilament suture of any of Embodiments 1-19, prepared via extruding a material comprising a PTFE resin to give an extruded PTFE and sintering the extruded PTFE by passing the extruded PTFE through one or more sintering ovens with a pulling speed of greater than 30 feet per minute.
Embodiment 21: The non-absorbable, monofilament suture of Embodiment 20, wherein the sintering is conducted at one or more temperatures in the range of 500° F. to 700° F.
Embodiment 22: The non-absorbable, monofilament suture of Embodiment 21, wherein no portion of the sintering is conducted at a temperature above 700° F.
Embodiment 23: The non-absorbable, monofilament suture of any of Embodiments 1-19, prepared via extruding a material comprising a PTFE resin; sintering the extruded PTFE by passing the extruded PTFE through one or more sintering ovens with a pulling speed of greater than 65 feet per minute to give a sintered monofilament; and drawing down the sintered monofilament to give a desired outer diameter.
Embodiment 24: The non-absorbable, monofilament suture of Embodiment 24, wherein the sintering is conducted at one or more temperatures in the range of 580° F. to 720° F.
Embodiment 25: A dental or cardiac suture, comprising the non-absorbable, monofilament suture of any of Embodiments 1-24.
Embodiment 26: A tether or anchor in a medical device, comprising the non-absorbable, monofilament suture of any of Embodiments 1-24.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

ePTFE sutures and/or microfilaments are provided herein, with particularly advantageous tensile properties for their respective sizes, as will be described herein in further detail. In particular, in some embodiments, ePTFE sutures and microfilaments are provided with enhanced knot pull strength, enhanced straight pull strength, and/or both enhanced knot pull and straight pull strengths relative to comparable known ePTFE sutures and microfilaments of the same size and composition. In some embodiments, the enhanced strength values are obtained by optimizing material processing parameters as disclosed herein below.

ePTFE is a porous form of poly(tetrafluoroethylene), characterized by nodes interconnected by fibrils. ePTFE can be prepared, e.g., according to the disclosure of U.S. Pat. No. 3,953,566 to Gore, which is incorporated herein by reference in its entirety. Briefly, a mixture comprising PTFE powder and a lubricant is extruded and stretched so as to provide a microstructure exhibiting nodes that are connected by fibrils, giving a material that is a softer, more flexible, and porous alternative to standard PTFE. ePTFE is advantageously inert, has a soft feel, high lubricity, and can provide for control of tissue ingrowth.

By “ePTFE suture” or “ePTFE microfilament” is meant that the suture or microfilament comprises a significant amount of ePTFE (e.g., at least about 50%, at least about 60%, at least about 70%, at least about 80%, at least about 90%, at least about 95%, at least about 98%, at least about 99%, at least about 99.5%, at least about 99.8%, or at least about 99.9% by weight, up to 100% ePTFE by weight). In some embodiments, such ePTFE sutures and/or microfilaments can consist of ePTFE or can consist essentially of ePTFE. In some embodiments, the ePTFE sutures and/or microfilaments can comprise one or more additional components. In some embodiments, the ePTFE sutures can include one or more agents to improve handling, antibacterial properties, and/or visibility (which can be associated with the suture material in various ways, including being impregnated within the ePTFE). Exemplary such additional agents include, but are not limited to, antimicrobial agents (antibiotic agents and/or antifungal agents), local anesthetics or analgesics, dyes/pigments, color stabilizers, Such additional agents typically do not contribute appreciably to the strength of the ePTFE sutures; as such, in some embodiments, the knot pull strengths and straight pull strengths reported herein are exhibited even in the absence of the optional additional components incorporated therein. In some embodiments, the ePTFE sutures and/or microfilaments described herein are uncoated and in some embodiments, they are coated (e.g., with a material that does not contribute appreciably to strength). In some embodiments, such coatings can be, e.g., waxes (e.g., beeswax, petroleum wax, polyethylene wax, etc.), silicone, fatty acid salts, silicone rubbers, poly(tetrafluoroethylene), polybutylate acid (PBA), polycaproloactones (alone or with calcium stearate), ethyl cellulose, or any combination thereof. In some embodiments, such coatings can comprise tie layers, which are polymeric materials (e.g., thermoplastics) as generally known in the art. In some embodiments, such coatings can comprise one or more of the additional agents referenced above, e.g., antimicrobial agents. In some embodiments, the knot pull strengths and straight pull strengths reported herein are exhibited even in the absence of the optional coating thereon.

Sutures can generally be single-strand sutures (also referred to as “monofilament” sutures, comprising a single microfilament) or multiple-strand sutures (also referred to as “multifilament” sutures, comprising two or more microfilaments that can be associated with one another, e.g., by spinning, twisting or braiding). The disclosure herein is limited to mono-filament sutures. The disclosed monofilament sutures described herein are typically not barbed. It is noted that the length of the sutures described herein is not particularly limited.

Surgical sutures (including the ePTFE sutures described herein) can be classified e.g., by their outer diameters (OD) as defined by the United States Pharmacopeia (USP) system. It is noted that sutures generally exhibit substantially uniform ODs along the length of the suture; as such, the “OD” values disclosed herein can, in some embodiments, be “average” OD values.

The USP system classifies suture OD values based, in part, on the values in the following table.

TABLE 1
USP Classifications of Sutures

| USP Classification | OD (mm)* |
|---|---|
| 5 | 0.700-0.799 |
| 4 | 0.600-0.699 |
| 3 | 0.600-0.699 |
| 2 | 0.500-0.599 |
| 1 | 0.400-0.499 |
| 0 | 0.350-0.399 |
| 2-0 | 0.300-0.349 |
| 3-0 | 0.200-0.249 |
| 4-0 | 0.150-0.199 |
| 5-0 | 0.100-0.149 |
| 6-0 | 0.070-0.099 |
| 7-0 | 0.050-0.069 |
| 8-0 | 0.040-0.049 |
| 9-0 | 0.030-0.039 |

*as measured by direct contact

In some embodiments, the disclosed sutures have a USP of 1, 0, or 2-0; in some embodiments, the disclosed sutures have a USP of 3-0, 4-0, or 5-0; in some embodiments, the disclosed sutures have a USP of 3; in some embodiments, the disclosed sutures have a USP of 2; in some embodiments, the disclosed sutures have a USP of 1; in some embodiments, the disclosed sutures have a USP of 0; in some embodiments, the disclosed sutures have a USP of 2-0; in some embodiments, the disclosed sutures have a USP of 3-0; in some embodiments, the disclosed sutures have a USP of 4-0; in some embodiments, the disclosed sutures have a USP of 5-0; and in some embodiments, the disclosed sutures have a USP of 6-0. The ePTFE sutures provided herein in some embodiments have an OD between 0.0084 inches (˜0.21 mm) and 0.03 1 inches (˜0.79 mm) or between 0.0075 inches (˜0.19 mm) and 0.031 inches (˜0.79 mm), which correlate (as shown above) to USP sizes 4-0, 3-0, 2-0, 1-0, 0, 1, 2, 3, 4, and 5. The OD values of the ePTFE sutures provided herein is measured in terms of non-contact OD via a laser micrometer. An alternative OD value is obtained as a USP OD, which can be recorded using a Mahr MarCator 1086 R, which measures OD by direct contact with the suture. The non-contact OD and USP OD often differ in that the USP OD generally has a lower value compared to the non-contact OD due to the material compression that occurs during the testing process. The density of the ePTFE suture in some embodiments has a value no more than 1.8 g/cc, e.g., no more than 1.79 g/cc, no more than 1.75 g/cc, no more than 1.70 g/cc, no more than 1.65 g/cc, or no more than 1.60 g/cc, with a minimum density of 0.75 g/cc. Such values, in some embodiments, allow for needles to be easily swaged onto the suture. Sutures that are produced using a drawdown process as referenced herein may have a higher density than sutures that have undergone only extrusion (with no drawdown after extrusion). The density of the ePTFE suture or microfilament is calculated first by weighing a 12-inch length of material and recording the weight in grams. The non-contact OD is then measured in inches via a laser micrometer as stated above. With this information, suture density can then be calculated using the following formula:

$$Density ( g / cc ) = \frac{Weight of Sample ( g )}{{π ( \frac{OD of Sample ( in )}{2} )}^{2} \times Length of Sample ( in ) \times 2.5 {4}^{3}}$$

The tensile strength values of the disclosed sutures or microfilaments are, as noted above, advantageously enhanced relative to known sutures or microfilaments of comparable size (e.g., OD) and composition. Tensile strength of a suture is the force that the suture will withstand before it breaks when knotted. In the context of the present disclosure, “straight pull strength” is a measure of the maximum force a suture can withstand before breaking when tension is applied to it in a straight, longitudinal line. Also known as “out of package” or “original” tensile strength, this value represents the full inherent strength of the suture material itself, before being affected by knots or tissue friction. In the context of the present disclosure, “knot pull strength” (or “knot pull tensile strength”) is a measure of the force required to break a knotted suture material. It differs from a standard tensile strength test in that the suture is knotted before being pulled, and the resulting strength can vary significantly due to the stresses and deformation introduced by the knot. This strength is affected, e.g., by the suture's diameter, material type, material processing, and knot design, which can introduce significant complexity to the relationship between straight pull strength and knot pull strength, especially across a broad range of low to high suture diameters. While various documents investigate enhancement of straight pull strength, these enhancements do not always lead to suitable knot pull strength and certainly do predictably result in optimized knot pull strength. As a general rule, the smaller the OD of a suture, the less tensile strength the suture will have. As such, in some embodiments, it is relevant to consider the tensile strength values of a given ePTFE suture relative to its OD.

According to the present disclosure, in some embodiments, ePTFE sutures or microfilaments exhibit higher maximum loads for knot pull strength than previously reported for such materials (referenced in the data presented herein as “average maximum load”). In some embodiments, the maximum load for knot pull strength of ePTFE sutures or microfilaments as provided herein has a value up to about 15 lbf, e.g., between 4.37 lbf and 14.30 lbf, which (as referenced herein above) can be dependent on the OD of the suture of microfilament.

The maximum load for knot pull strength can be determined by conducting a tensile test using an Instron tensile tester with Bluehill 3 software. Testing is conducted according to USP 881 using a 10 lb load cell. The instrument is set so that the gage length is 6 inches and moves at a rate of 10 inches per minute. An ePTFE suture sample with length exceeding 6 inches is secured in the top pneumatic grip. A knot is then tied in the middle of the sample such that the knot is located approximately equidistant to each of the grips. The loose end of the sample is then secured in the bottom pneumatic grip. The software is then engaged and records the maximum load values. At least 10 replicates are performed for each material sample. Average maximum loads as reported herein are the average of the replicates.

According to the present disclosure, in some embodiments, ePTFE sutures or microfilaments exhibit higher maximum load for straight pull strength than previously reported for such materials. In some embodiments, the maximum load straight pull strength of ePTFE sutures or microfilaments as provided herein has a value of up to about 15 lbf, e.g., up to about 12 lbf or up to about 10 lbf, and in particular embodiments, between 4.03 and 7.72 lbf,

The maximum load straight pull strength can be determined by conducting a tensile test using an Instron tensile tester with Bluehill 3 software. Testing is conducted according to USP 881 using a 10 lb load cell. The instrument is set so that the gage length is 10 inches and moves at a rate of 12 inches per minute. An ePTFE suture sample with length exceeding 10 inches is secured in the top and bottom pneumatic grips. The software is then engaged and records the maximum load values. At least 10 replicates are performed for each material sample.

In general, some materials may have a high straight pull strength but a weak knot pull strength, e.g., as illustrated in Comparative Example 1 (straight pull strength of 11.5 lbf; knot pull strength 7.5 lbf). The relationship between these two tensile properties is not directly proportional, and can be unpredictable if relying solely on the measure of straight pull strength as an indicator of knot pull strength. In some embodiments, the knot pull strength of the disclosed ePTFE sutures and microfilaments is comparable to their straight pull strength. In some embodiments, the knot pull strength of the disclosed ePTFE sutures and microfilaments is greater than their straight pull strength. In some embodiments, the knot pull strength of the disclosed ePTFE sutures and microfilaments is less than their straight pull strength. In some embodiments, the drawdown process increases the knot pull strength of the disclosed ePTFE sutures and microfilaments. The disclosed ePTFE sutures and microfilaments can be used in a range of applications, particularly in the medical field. For example, in various embodiments, the ePTFE sutures can be used as sutures for dental or cardiac applications. In some embodiments, ePTFE sutures can be used as tethers or anchors in medical devices (e.g., as used in procedures such as aortic repair or mitral valve repair). These applications may require a higher knot pull strength than currently provided by ePTFE monofilament sutures on the market, which higher knot pull strengths are, in some embodiments, uniquely exhibited by the ePTFE sutures described herein.

The disclosed ePTFE sutures and microfilaments are generally prepared by providing an ePTFE material (e.g., as referenced above), extruding the ePTFE material, and sintering the resulting filament. Parameters such as temperatures, reduction ratio, ram speed, and pulling speed may be altered to increase knot pull strength at a given OD to obtain sutures and microfilaments with the enhanced tensile strength values outlined herein (as described in further detail herein below). The OD of each suture highly influences the maximum load knot pull strength, and for sutures with certain low desired OD values, e.g., an OD less than 0.012 inches (˜3.05 mm), a secondary drawdown process may be required to achieve both a small diameter and sufficient knot pull strength. This drawdown can be conducted by processing an extruded monofilament through heated dies such that the diameter is reduced without compromising tensile properties.

In some embodiments, the disclosed monofilaments are described by their average knot pull strength relative to diameter (AKP/OD value). The inventors have discovered that, in order to increase the AKP/OD, the key processing parameters were the pull speed post-sintering and the temperature zones within the sintering ovens for the ePTFE sutures. Conventionally, when straight pull strength is being maximized, the pull speed is reduced to less than 30 feet per minute (fpm) with 23-27 fpm being optimal in combination with a higher temperature profile of sintering (e.g., with higher temperatures within one or more independent temperature zones of one or more sintering ovens, such as six independent temperature zones when two sintering ovens are used). Typical temperatures range from about 600-800° F., preferably about 675-790° F., where the lower end of the range represents temperatures toward the end of the sintering arrangement, e.g., at the back end of the second sintering oven that experiences greater exposure to ambient air (about 600-610° F.), and the higher end of the range represents temperatures in zones of the oven more towards the front and center or areas less exposed to ambient air (about 715-800° F.).

Such processing parameters can result in excellent straight pull strength as shown in Comparative Example 1 (ASP=11.5 lbF). However, the AKP/OD for this sample is significantly lower (250) than what can be achieved if the process parameters are modified in a nonconventional way in order to enhance the AKP/OD as discovered by the inventors. In order to maximize the AKP/OD, the pull speed (through the sintering oven) is advantageously greater than 30 fpm, preferably 35-48 fpm as was the case for Example 1 testing for the AHSM size 4 and the AHSM size 3 ePTFE sutures. One of the reasons this was previously thought to be suboptimal is because increasing pull speeds to that range significantly increases the risk of breaks for a given OD of ePTFE suture. However, the inventors have found that, when the temperature profile is lowered significantly with respect to the conventional temperatures ranges referenced above, e.g., to a range of about 500-700° F., preferably about 565-675° F., where the lower end of the range represents temperatures at the back end of the second sintering oven that experiences greater exposure to ambient air (about 565-580° F.), and the higher end of the range represents temperatures in zones of the oven more towards the front and center or areas less exposed to ambient air (about 600-675° F.), this results in excellent AKP/OD values up to about 499, e.g., as seen in Example 1 for AHSM size 3 and up to about 419 as seen in Example 1 for AHSM size 4. Advantageously, such a process can, in some embodiments, be conducted such that the ePTFE is not subjected to temperatures higher than about 750° F., not subjected to temperatures higher than about 725° F., or not subjected to temperatures higher than about 710° F. during this process (while typical processes experience such temperatures in at least one zone within the sintering oven).

When the ePTFE sutures are to be further drawn down, different parameters are relevant. As demonstrated in Comparative Example 3 and Example 2 (for AHSM 5 the smallest diameter suture tested), this unconventional relationship between pull speed and temperature profile processing parameters was employed to obtain to the highest AKP/OD achieved (up to about 520 for Example 2), which was significantly higher than what was achieved for Comparative Example 3 (up to about 125). The pull speed for Comparative Example 3 was 62 fpm, while the temperature profile of the sintering oven zones ranged from about 650-775° F. By contrast, the pull speed for Example 2 was 70 fpm, while the temperature profile of the sintering oven zones ranged from about 580-720° F. Again the higher pull speed and lower temperature profile of the sintering ovens enabled a significantly higher AKP/OD to be achieved. Advantageously, such a process can, in some embodiments, be conducted such that the ePTFE is not subjected to temperatures higher than about 750° F., not subjected to temperatures higher than about 740° F., or not subjected to temperatures higher than about 720° F. during this process (while typical processes experience such temperatures in at least one zone within the sintering oven).

Aspects of the present invention are more fully illustrated by the following examples, which are set forth to illustrate certain aspects of the present invention and are not to be construed as limiting thereof.

### EXAMPLES

### Comparative Example 1

An ePTFE suture was extruded from fine powder PTFE resin via ram extrusion. Upon characterizing the suture using a laser micrometer, the average non-contact OD was found to be 0.030 inches. A tensile test measuring knot pull strength was conducted using an Instron and found that the average maximum load was 7.50 lbf. Additionally, a tensile test measuring straight pull strength was conducted using an Instron and found that the average maximum load was 11.50 lbf. A 12-inch sample of material was weighed and recorded in grams. Using this value along with the OD measurement, the density was calculated to be 0.75 g/cc.

### Comparative Example 2

An ePTFE suture was extruded from fine powder PTFE resin via ram extrusion. Upon characterizing the suture using a laser micrometer, the average non-contact OD was found to be 0.0156 inches. A tensile test measuring knot pull strength was conducted using an Instron and found that the average maximum load was 4.28 lbf. Additionally, a tensile test measuring straight pull strength was conducted using an Instron and found that the average maximum load was 5.10 lbf. A 12-inch sample of material was weighed and recorded in grams. Using this value along with the OD measurement, the density was calculated to be 0.92 g/cc.

### Comparative Example 3

An ePTFE suture was extruded from fine powder PTFE resin via ram extrusion, then was secondarily drawn down using heated dies to reduce the OD of the suture. Upon characterizing the suture using a laser micrometer, the average non-contact OD after the drawdown process was found to be 0.0088 inches. A tensile test measuring knot pull strength was conducted using an Instron and found that the average maximum load was 1.10 lbf. Additionally, a tensile test measuring straight pull strength was conducted using an Instron and found that the average maximum load was 1.80 lbf. A 12-inch sample of material was weighed and recorded in grams. Using this value along with the OD measurement, the density was calculated to be 1.21 g/cc.

### Example 1

An ePTFE suture was extruded from fine powder PTFE resin via ram extrusion. Upon characterizing the suture using a laser micrometer, the average non-contact OD was found to be 0.015 inches. A tensile test measuring knot pull strength was conducted using an Instron and found that the average maximum load was 7.48 lbf. Additionally, a tensile test measuring straight pull strength was conducted using an Instron and found that the average maximum load was 7.57 lbf. A 12-inch sample of material was weighed and recorded in grams. Using this value along with the OD measurement, the density was calculated to be 1.00 g/cc. This example displays an improvement in tensile strength compared to both Comparative Examples 1 and 2. The suture of Example 1 was able to achieve a similar tensile strength to Comparative Example 1 with a smaller OD and a significantly heightened tensile strength compared to the suture of similar OD in Comparative Example 2. This shows that through changes in process parameters during the extrusion process, tensile strength can be improved at a given OD without necessarily impacting the density of the suture.

### Example 2

An ePTFE suture was extruded from fine powder PTFE resin via ram extrusion. Upon characterizing the suture using a laser micrometer, the average non-contact OD after the drawdown process was found to be 0.012 inches. A tensile test measuring knot pull strength was conducted using an Instron and found that the average maximum load was 4.20 lbf. Additionally, a tensile test measuring straight pull strength was conducted using an Instron and found that the average maximum load was 4.03 lbf. A 12-inch sample of material was weighed and recorded in grams. Using this value along with the OD measurement, the density was calculated to be 0.93 g/cc. Then, the sample was secondarily drawn down using heated dies to reduce the OD of the suture. The average non-contact OD, maximum load knot pull strength, and density were recalculated after this process to be 0.0084 inches, 4.37 lbf, and 1.79 g/cc, respectively.

This example shows that with standard extrusion conditions, the suture of Comparative Example 3 is limited in the tensile strength it can achieve even through the drawdown process. If tensile strength is attempted to be improved beyond what was achieved in this Comparative Example, the suture would typically change significantly in OD outside of the desired OD range or break during the drawdown process. With the suture in Example 2, extrusion conditions were able to be manipulated so that the tensile strength could improve at a given OD.

For all of the examples listed, extrusion parameters such as temperatures, reduction ratio, ram speed, and pulling speed were able to be manipulated beyond the standard extrusion procedures to improve both knot pull and straight pull tensile strength. For suture sizes that do not require a drawdown step, both the density and OD were often able to be maintained while increasing tensile strength. Suture sizes that do require a drawdown step did not necessarily have similar densities to the respective comparative suture (Comparative Example 3), but OD was able to be maintained throughout the drawdown process while significantly increasing tensile strength. Under standard extrusion conditions, the suture often breaks during the drawdown process when trying to improve tensile strength, thus showing that the process conditions altered during the above examples are the significant driving factor in increasing tensile strength of ePTFE sutures. As illustrated by the comparative examples, these increases in knot pull strength far exceed what is typically seen in the field.

For each comparative example and example below, results are summarized in Tables 2 and 3. Table 2 displays results from sutures with USP Sizes 1-4, whereas Table 3 shows results from USP Size 5 sutures. Beyond the examples below, additional data points for USP Size 3, 4, and 5 sutures are also provided. The data points specifically mentioned in the Examples are bolded in each table. Not all data points are fully characterized.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A non-absorbable, monofilament suture comprising expanded polytetrafluoroethylene (“ePTFE”), wherein:
the non-absorbable, monofilament suture has a non-contact outer diameter (“OD”) of 0.016 inches or less, and an average knot pull strength in lbf, divided by the OD (“AKP/OD”), of 3884 or greater; and/or
the non-absorbable, monofilament suture has a non-contact outer diameter (“OD”) of 0.018 inches or less, and an average straight pull strength in lbf, divided by the OD (“ASP/OD”), of 400 or greater.

2. The non-absorbable, monofilament suture of claim 1, wherein the AKP/OD is 350 or greater.

3. The non-absorbable, monofilament suture of claim 2, wherein the AKP/OD is 400 or greater.

4. The non-absorbable, monofilament suture of claim 1, wherein the ASP/OD is 400 or greater.

5. The non-absorbable, monofilament suture of claim 4, wherein the ASP/OD is 500 or greater.

6. The non-absorbable, monofilament suture of claim 1, wherein the OD is 0.018 inches or less, and wherein the AKP/OD is 350 or greater and the ASP/OD is 0.40 or greater.

7. The non-absorbable, monofilament suture of claim 1, wherein the average knot pull strength in lbf is 15 lbf or less.

8. The non-absorbable, monofilament suture of claim 7, wherein the average knot pull strength in lbf is 14.3 lbf or less.

9. A non-absorbable, monofilament suture comprising expanded PTFE (“ePTFE”), selected from the group consisting of:
a non-absorbable, monofilament suture comprising ePTFE with a USP diameter of 5-0 and a knot pull strength of at least 3.0 lbf or at least 3.2 lbf;
a non-absorbable, monofilament suture comprising ePTFE with a USP diameter of 4-0 and a knot pull strength of at least 4.8 lbf;
a non-absorbable, monofilament suture comprising ePTFE with a USP diameter of 3-0 and a knot pull strength of at least 6 lbf or at least 7 lbf;
a non-absorbable, monofilament suture comprising ePTFE with a USP diameter of 2-0 and a knot pull strength of at least 7 lbf or at least 8 lbf;
a non-absorbable, monofilament suture comprising ePTFE with a USP diameter of 0 and a knot pull strength of at least 9 lbf, at least 10 lbf, or at least 11 lbf; and
a non-absorbable, monofilament suture comprising ePTFE with a USP diameter of 1 and a knot pull strength of at least 13 lbf or at least 14 lbf.

10. The non-absorbable, monofilament suture of claim 1, wherein the knot pull strength is 15 lbf or less.

11. The non-absorbable, monofilament suture of claim 1, wherein the knot pull strength is 14.3 lbf or less.

12. A non-absorbable, monofilament suture comprising expanded PTFE (“ePTFE”), selected from the group consisting of:
a non-absorbable, monofilament suture comprising ePTFE with a USP diameter of 5-0 and a knot pull strength of at least 3.0 lbf or at least 3.2 lbf; and
a non-absorbable, monofilament suture comprising ePTFE with a USP diameter of 3-0 and a knot pull strength of at least 6 lbf or at least 7 lbf.

13. The non-absorbable, monofilament suture of claim 12, wherein the knot pull strength is 15 lbf or less.

14. The non-absorbable, monofilament suture of claim 13, wherein the knot pull strength is 14.3 lbf or less.

15. The non-absorbable, monofilament suture of claim 1, consisting essentially of ePTFE.

16. The non-absorbable, monofilament suture of claim 1, comprising one or more components associated with the ePTFE, wherein the one or more components are selected from the group consisting of agents to improve handling, antibacterial properties, and/or visibility.

17. The non-absorbable, monofilament suture of claim 1, wherein the suture is uncoated.

18. The non-absorbable, monofilament suture of claim 1, wherein the non-absorbable monofilament suture has a density of 1.8 g/cc or less.

19. The non-absorbable, monofilament suture of claim 18, wherein the density is 1.60 g/cc or less.

20. The non-absorbable, monofilament suture of claim 1, prepared via extruding a material comprising a PTFE resin to give an extruded PTFE and sintering the extruded PTFE by passing the extruded PTFE through one or more sintering ovens with a pulling speed of greater than 30 feet per minute.

21. The non-absorbable, monofilament suture of claim 20, wherein the sintering is conducted at one or more temperatures in the range of 500° F. to 700° F.

22. The non-absorbable, monofilament suture of claim 1, prepared via extruding a material comprising a PTFE resin; sintering the extruded PTFE by passing the extruded PTFE through one or more sintering ovens with a pulling speed of greater than 65 feet per minute to give a sintered monofilament; and drawing down the sintered monofilament to give a desired outer diameter.

23. The non-absorbable, monofilament suture of claim 22, wherein the sintering is conducted at one or more temperatures in the range of 580° F. to 720° F.

24. A dental or cardiac suture, comprising the non-absorbable, monofilament suture of claim 1.

25. A tether or anchor in a medical device, comprising the non-absorbable, monofilament suture of claim 1.
